(19) United States
(12) Patent Application Publication
(10) Pub. No.: US 2010/0331415 A1
(43) Pub. Date: Dec. 30, 2010

(54) USE OF OMEGA-3-FATTY ACID(S)

(75) Inventors: Rudolf Krumbholz, Merchweiler (DE); Karin Krumbholz, Merchweiler (DE); Christine Krumbholz, Merchweiler (DE); Norbert Schirra, Voelklingen (DE); Peter Lembke, Gersheim-Reinheim (DE)

Correspondence Address: COLLARD & ROE, P.C., 1077 NORTHERN BOULEVARD, ROSLYN, NY, 11576, US

(73) Assignee: K. D. PHARMA BEXBACH GMBH, BEXBACH (DE)

(21) Appl. No.: 12/734,657

(22) PCT Filed: Nov. 18, 2008
(86) PCT No.: PCT/DE2008/001928

§ 371 Date: May 14, 2010

(30) Foreign Application Priority Data
Nov. 19, 2007 (DE) 10 2007 055 344.9

(51) Int. Cl. A61K 31/20; C07C 57/03; A61P 29/00

(52) U.S. Cl. 514/560; 554/224

(57) ABSTRACT

The invention relates to the use of omega-3-fatty acid(s) in means for pain relief. The means preferably comprise highly unsaturated omega-3-fatty acid(s) that contain, above all, EPA and/or DHA. Preferably, the omega-3-fatty acid is administered in a high dose of more than 2 g, in particular more than 3 to 4 g, for pain relief.

The invention relates to a novel use of omega-3 fatty acid(s).

It has been known for some time from the technical literature that inflammation-inhibiting and cardioprotective effects proceed from omega-3 fatty acid(s), particularly highly unsaturated omega-3 fatty acid(s), above all EPA and DHA. Positive effects can already be achieved when EPA and DHA are taken daily at doses between 0.5 and 1 g over several weeks. After treatment with a gram of EPA and DHA per day over six months, it was possible to determine a noticeable reduction in the risk of cardiac infarction (G. Ital. Cardiol. 1993; 23:1053-1061). Treatment of painful inflammations and rheumatic illnesses with omega-3 fatty acid(s) also shows positive effects after at least a month (Alair A. B., et al., Nutrition (2005); 1, 2, 131-136). Furthermore, the use of omega-3 fatty acids for long-term therapy of dermatitis and psoriasis (J. Am. Acad. Dermatol. (1998); 38, 539-547) and for the treatment of various premenstrual complaints (Deutsch B. et al., Nutrition Research (2000); 5 (20), 621-631 [11]) are known.

The invention is based on the task of opening up further possibilities for use of omega-3 fatty acid(s).

According to the invention, omega-3 fatty acid(s) are used in a remedy for combating pain.

The inventor has recognized that omega-3 fatty acids are suitable not only for the known effects described above, but also directly for relief of acute pain, and in this regard also develop a short-term effect that sets in within hours.

Highly unsaturated fatty acids, above all EPA (eicosapentanoic acid) and DHA (docosahexanoic acid) are particularly effective in combating pain.

An effect as a pain reliever that starts in the short term particularly occurs at high dosages of the omega-3 fatty acid(s), for example at a dosage amount of more than 2 g, particularly of more than 3 to 4 g.

Preferably, the entire dosage amount is taken at one time in order to combat pain.

The mechanism of action of the pain relief has commonalities with that of the active ingredient of aspirin. The intake of omega-3 fatty acid(s), particularly at high dosages, reduces or competitively blocks the production of inflammation-promoting substances that cause pain, particularly tissue hormones, from omega-6 fatty acid(s), by way of the COX enzyme.

At the same time, inflammation-inhibiting substances are formed from the administered omega-3 fatty acid(s) itself/themselves, by way of the same COX enzyme.

Preferably, the preparation that contains the omega-3 fatty acid(s) is free of or low in other fatty acids, particularly omega-6 fatty acids.

While the omega-3 fatty acid(s) in the pain reliever can be combined with at least one other active substance, the preparation, in a preferred embodiment of the invention, contains exclusively omega-3 fatty acid(s) and/or compounds of the omega-3 fatty acid(s) as active substances. Flavorings are possible as additives.

The preparation that contains the omega-3 fatty acid(s) can be used for combating pain, for example in the case of inflammations or injuries.

Among other things, the preparation can be used to combat headaches, toothaches, joint pain, menstrual complaints, or muscle soreness.

It is advantageous that the preparation can be used in preventive manner, particularly for muscle soreness pain, for example after extended periods of non-exercise before resuming exercise. The preventive effect against muscle soreness pain is furthermore based on an increase in the elasticity of the cells in the so-called Z-disk and a related reduction in the formation of tears. Taking omega-3 fatty acid(s) shortly before an above-average demand on the muscles, for example before a tennis tournament, prevents or markedly reduces the formation of muscle soreness.

The effect of the preparation will be described below, using several examples.

### 1^{ST} EXAMPLE

The effect against pain of varying types, above all headaches, was tested in a large number of test subjects. In the case of one-time administration of 3 g highly unsaturated omega-3 fatty acid(s), in each instance (product PUR3 from K.D. Pharma Bexbach GmbH: 60% EPA+20% DHA+10% other omega-3 fatty acids), a noticeable decrease in pain was achieved within one day, in the case of headache actually within a few hours.

### 2^{ND} EXAMPLE

The applicant investigated the preventive effect of omega-3 fatty acid(s) for muscle soreness, using himself, among others, as a test subject. The administration of 3 g omega-3 fatty acid(s) (product from K.D. Pharma Bexbach GmbH: 70% EPA+12% DHA) one day before demanding muscle use (gymnastic exercise over a long period of time, hiking in the mountains, garden work) prevented the occurrence of muscle soreness in all the cases observed, entirely or in part. The effect decreases if the preparation is taken earlier than three days before the activity.

### 3^{RD} EXAMPLE

An amount of omega-3 fatty acid(s) (K.D. Pharma—product: 39% EPA+43% DHA) was taken in the morning before a tennis tournament that began at 2:00 p.m., by ten persons. For all of these persons, the tournament represented muscle activity that more or less went beyond the usual extent of their sports activity in terms of intensity and duration, and would have led to muscle soreness, according to their experience. However, no noteworthy muscle soreness occurred in any of the persons in question.

1. Use of omega-3 fatty acid(s) in a preparation for combating pain.

2. Use according to claim 1, wherein
the omega-3 fatty acid(s) comprise highly unsaturated omega-3 fatty acid(s).

3. Use according to claim 2, wherein
the omega-3 fatty acid(s) comprise EPA, DHA, DPA, ETA, 21:5ₙ3, and/or SDA.

4. Use according to claim 1, wherein
the omega-3 fatty acid(s) are used as free fatty acids or in bonded form, particularly as esters or salts.

5. Use according to claim 1, wherein
the omega-3 fatty acid(s) are administered to combat pain, in a dosage of more than 2 g, preferably 3 to 4 g.

6. Use according to claim 1, wherein
a specific dosage amount of the preparation is administered once, to combat pain.

7. Use according to claim 1, wherein
the preparation is free of or low in other fatty acids.

8. Use according to claim 1, wherein
the preparation contains the omega-3 fatty acid(s) alone, as a pain-relieving substance, or contains the omega-3 fatty acid(s) in combination with other substances, particularly pain-relieving active substances.

9. Use according to claim 8, wherein
the preparation contains a flavoring as another substance.

10. Use according to claim 1, wherein
the preparation for combating pain is used for inflammations or/and injuries.

11. Use according to claim 1, wherein
the preparation is used to combat headaches, toothaches, joint pain, menstrual complaints or/and muscle soreness.

12. Use according to claim 1, wherein
the preparation is used in preventive manner, particularly to prevent muscle soreness.

13. Use according to claim 1, wherein
the omega-3 fatty acid(s) are administered intravenously or orally.

14. Use according to claim 13, wherein
the omega-3 fatty acid(s) are administered in the form of capsules, tablets or/and liquids.
